(19) United States
(12) Patent Application Publication
(10) Pub. No.: US 2022/0361606 A1
(43) Pub. Date: Nov. 17, 2022

(54) GARMENT FOR INTERFACING A MEDICAL DEVICE WITH AN IMPLANT

(71) Applicant: Syril A. Strickler, Camarillo, CA (US)

(72) Inventor: Syril A. Strickler, Camarillo, CA (US)

(21) Appl. No.: 17/733,472

(22) Filed: Apr. 29, 2022

Related U.S. Application Data
(60) Provisional application No. 63/188,968, filed on May 14, 2021.

(51) Int. Cl. A41D 27/20

(52) U.S. Cl. CPC A41D 27/205; A41B 2300/32

(57) ABSTRACT

The disclosure relates to garments for holding a medical device and interfacing same with an implant or a second device disposed on or within a body of a user so as to align and enable communication therebetween.

### CROSS-REFERENCE TO RELATED APPLICATIONS

This application claims the benefit of priority under 35 U.S.C. § 119(e) of U.S. Provisional Patent Application No. 63/188,968, entitled GARMENT FOR INTERFACING A MEDICAL DEVICE WITH AN IMPLANT, filed on May 14, 2021.

### TECHNOLOGICAL FIELD

The present disclosure generally relates to the field of medical implants, and in particular, interfacing auxiliary devices with the medical implant.

### BACKGROUND

Certain types of implants, such as a neurotransmitter, require a user (i.e., patient) to interface a complimentary or auxiliary piece of equipment in proximity with the implant for charging, activating, exchanging data, etc. and maintain the equipment in communication with the implant for a certain amount of time. Doing so can be difficult for the user, especially where the equipment must be maintained in a specific position for a long period of time and/or the equipment is cumbersome (heavy, large, oddly shaped, etc.), or the patient is in a weakened or otherwise compromised state.

Thus, advances with respect to interfacing auxiliary equipment, such as an electronic device, with a medical implant would be desirable.

### BRIEF SUMMARY

Example implementations of the present disclosure are directed to the concept of a garment for holding and interfacing an electronic device with a medical device implanted within a body.

In one aspect, the disclosure provides for a garment for interfacing an electronic device with a medical implant. The garment includes an envelope made from a textile material and configured to at least partially surround or contact a portion of a human body where the implant is located and a receptacle disposed on or within the envelope, where the receptacle is configured to removably secure the electronic device to the envelope and in proximity to the portion of a human body where the implant is located. The receptacle allows for the operative alignment of the electronic or a secondary medical device with the implant.

In another aspect, the disclosure provides for a garment for interfacing an electronic device with a medical device implanted within a patient's head. The garment includes an envelope made from a textile material and configured to at least partially surround the patient's head and a receptacle coupled to the envelope and configured to removably secure the electronic device thereto, where the receptacle is disposed on or within the envelope so as to operatively align the electronic device with the medical device. In some embodiments of this aspect, the electronic device is a receiver/transmitter for downloading data from the implant and transmitting the data to an intended recipient, such as, for example, wirelessly to a physician or technician, or storing the data on to a fixed or portable drive.

In yet another aspect, the disclosure is directed to a method of interfacing an electronic device with a medical implant disposed within a body. The method includes the steps of providing a garment made from a textile material and configured to at least partially surround a portion of the body, wherein the garment includes a receptacle configured to removably secure the electronic device at least partially therein, disposing the garment about the body, and orienting the garment relative to the body portion where the implant is located to operatively align the electronic device with the medical device.

In some example implementations of the garment of any preceding example implementation, or any combination of any preceding example implementations, the receptacle is defined by a partially open perimeter and may be accessible via an interior cavity or surface of the envelope and/or an exterior surface of the envelope. In some embodiments, the receptacle may be formed by two or more straps (with or without adjustability) that are sufficient to securely support the device in the proper position. In some embodiments, the envelope comprises a first panel and a second panel and the receptacle is defined therebetween. The first panel may be configured to be in closer proximity to the human body than the second panel, and the second panel may define an opening therethrough configured to receive the electronic device. In some embodiments, the first panel comprises an elastic mesh or netting and the second panel comprises a soft fabric attached to the first panel.

In some additional example implementations of the garment, the envelope may include at least one adjustment mechanism configured to accommodate the garment to differently sized and/or shaped body parts. The adjustment mechanism may include at least one of a hook and loop fastener (Velcro®), buttons, snaps, buckles, draw strings, zippers, or elastic fibers (e.g., additional elastic fibers woven into the base of the garment to provide a snug fit). Additionally, the envelope may be made from at least one of a soft fabric, a polymeric material, or a transparent or translucent material, such as those described herein. In some embodiments, a portion of the envelope and/or receptacle may be made of a material configured to allow for the passage of electromagnetic radiation therethrough and/or a portion of the envelope and/or receptacle may be made of a material configured to prevent the passage of electromagnetic radiation therethrough.

In various embodiments of the garment, the envelope is embodied in at least one of a hat, a shirt, a vest, a sleeve, a pair of pants, or a wrap. The envelope may include at least one closure or attachment mechanism configured to removably secure the garment to a patient, such as at least one of a hook and loop fastener (Velcro®), buttons, snaps, buckles, draw strings, zippers, or elastic fibers.

Additionally, the receptacle may be secured to the envelope via at least one of stitching, bonding, hook and loop fastener (Velcro®), buttons, snaps, or zippers, and the electronic device may be secured to or within the receptacle via at least one of stitching, hook and loop fastener (Velcro®), buttons, snaps, zippers, or a frictional fit. Generally, the receptacle is sized and shape to receive at least one of a receiver, a transmitter, an amplifier, a power source, a digital storage device, a display, or a monitor. In various embodiments, the receptacle is configured to provide positional adjustability to the electronic device to provide for an optimal operative alignment with the implant. In some embodiments, the garment may include the electronic device, such as, for example, a receiver, a transmitter, an amplifier, a power source, a digital storage device, a display, or a monitor.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURES

Having thus described aspects of the disclosure in the foregoing general terms, reference will now be made to the accompanying figures, which are not necessarily drawn to scale, and wherein:

FIG. 1 is a schematic side view of one example of a garment configured for interfacing an electronic device with an implant in accordance with one or more embodiments of the disclosure;

FIG. 2 is a schematic perspective view of an alternative example of a garment configured for interfacing an electronic device with an implant in accordance with one or more embodiments of the disclosure;

FIG. 3 is a schematic plan view of yet another alternative example of a garment configured for interfacing an electronic device with an implant in accordance with one or more embodiments of the disclosure;

FIGS. 4A and 4B are schematic plan and sectional views of a portion of the garment of any of the previous figures depicting the structure of the garment and how the electronic device is disposed therein in accordance with one or more embodiments of the disclosure; and

FIGS. 5A and 5B are schematic plan and sectional views of a portion of the garment of any of the previous figures depicting an alternative structure of the garment and how the electronic device is disposed therein in accordance with one or more embodiments of the disclosure.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. Like reference numerals refer to like elements throughout.

Unless specified otherwise or clear from context, references to first, second or the like should not be construed to imply a particular order. A feature described as being above another feature (unless specified otherwise or clear from context) may instead be below, and vice versa; and similarly, features described as being to the left of another feature else may instead be to the right, and vice versa. Also, while reference may be made herein to quantitative measures, values, geometric relationships or the like, unless otherwise stated, any one or more if not all of these may be absolute or approximate to account for acceptable variations that may occur, such as those due to engineering tolerances or the like.

As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true. Further, the articles “a” and “an” mean “one or more,” unless specified otherwise or clear from context to be directed to a singular form. Furthermore, it should be understood that unless otherwise specified, the terms “data,” “content,” “digital content,” “information,” and similar terms may be at times used interchangeably. Additionally, where multiples of the same components are described, the multiples may be referred to individually (e.g., ##a, ##b, ##c, etc.) or collectively (##). In addition, any dimensions shown in the figures are in English units and are for exemplary purposes only, and the actual dimensions will vary to suit a particular application.

Example implementations of the present disclosure are generally directed to garments (e.g., headwear, shirt, vest, sleeve, pants, footwear, belt/wrap, etc.) for interfacing an electronic device or a complimentary/secondary medical device with a medical device implanted within a patient. The devices may include a neurotransmitter, a power source (e.g., for recharging), or other data exchange devices that may, for example, download performance data from the implant, upload instructions, provide for manual actuation, etc. The devices may be wired or wireless. The implants may include, for example, e-stimulators (e.g., deep brain, gastric), cochlear, orthopedic, pacemaker, defibrillator, drug delivery (e.g., insulin pump), intraocular, and prosthetics.

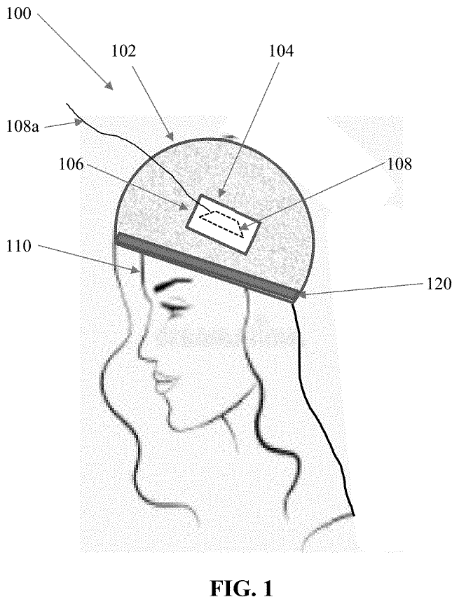

FIG. 1 depicts one embodiment of a garment 100, in this case a “beanie” type of headwear, although other types of “hats” are contemplated and considered within the scope of the invention. The garment 100 is made up of an envelope 102 that includes a textile material and is configured at least partially surround or otherwise contact a body part of a patient 110. As shown in FIG. 1, the body part is the patient's head; however, essentially any body part can be engaged with the garment 100, such as a torso, leg, arm, waist, foot, etc.

In a particular embodiment, the garment 100 is a type of hat that is configured to position a “wand” 108 adjacent to a deep brain stimulator (e.g., an RNS® neuro-stimulator from NeuroPace) so as to operatively engage the wand and stimulator to, for example, download a day's (or other time period) worth of activity and/or upload instructions to the stimulator to fine-tune the device to the particular patient. The garment 100 may have an inner panel or layer formed as a grid of elastic material for holding the device in a particular position and orientation to operatively engage the implant, either with or without additional structure (e.g. Velcro® straps for further securing the device 108). The garment 100 may also have an outer panel or layer of fabric (e.g., fleece) that is attached to and substantially covers the inner panel and may include a strong elastic band about the base of the outer layer to help secure the garment 100 to the head of the patient 110. Once the garment 100 is donned, the operation of the device 108 may proceed hands-free.

Generally, textile materials include a soft fabric (natural or synthetic fibers) that may be disposable or washable and/or a polymeric material. In some embodiments, some portions of the envelope 102 may be made of a soft, absorbent fabric (either disposable or washable), while another portion may be made of a transparent or translucent polymeric material (e.g., a soft vinyl or silicone) that makes it easier to view the placement of the device 108. In certain embodiments, the envelope material may be configured to allow electromagnetic radiation (EMR) to pass through one portion thereof, but not another portion of the envelope 102. For example, the material of the envelope 102 corresponding to where the device 108 interfaces with the implant may enable or otherwise promote the exchange of wireless communication (e.g., radio waves), while other portions of the envelope 102 may include a material (e.g., shielding) to prevent, for example, ambient electromagnetic interference from compromising the function of the device and/or implant. In some embodiments, the envelope material may include materials using elastic fibers, such as those sold by DuPont under the registered trademark Lycra®, which can be used to ensure a close contact between the garment 100 and the body part of the patient 110. Additionally, the material may include a high density material, a laminated material, a coated material including a material with a liquid coating, or a material having layers connected with glue points. Various portions of the envelope 102 may include materials that differ in air permeability, moisture wicking, thickness, flexibility, strength, or the like to suit a particular application.

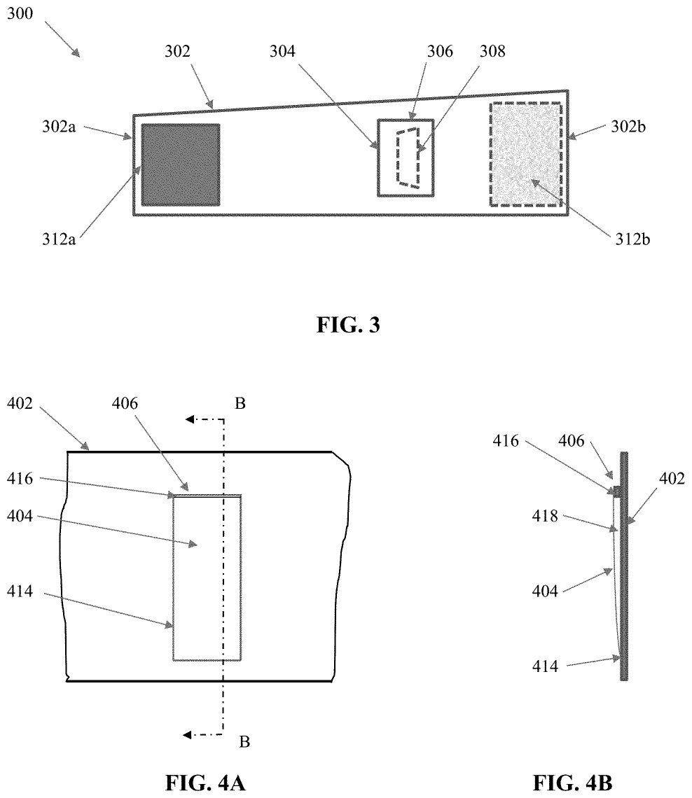

The garment 100 further includes a pocket or receptacle 104 coupled with the envelope 102 and configured to receive an electronic or medical device 108. The receptacle 104 essentially comprises one or more pieces of material, such as those disclosed herein, coupled to a surface of the envelope 102 via stitching (or other means as described herein) to define a receptacle cavity (e.g., inner cavity 418 in FIG. 4B). In one embodiment, the receptacle 104 has a generally rectangular shape and is stitched to the envelope 102 about a substantial portion of its perimeter (e.g., three sides thereof) leaving an open portion 106 of the perimeter (e.g., one side or end thereof) to allow the device 108 to be inserted and/or removed therefrom. In some cases, the open end 106 may be closeable to removably secure the device within the receptacle 104 by, for example, a hook and loop fastener (Velcro®), buttons, snaps, buckles, draw strings, zippers, elastic fibers, or other mechanisms as would be known to those in the apparel and/or medical clothing industry. Alternatively or additionally, the cavity of the receptacle may provide a frictional fit to the device 108 to further secure the device therein (e.g., in a specific orientation for optimal interfacing). Furthermore, the size and shape of the receptacle 104 will vary to suit a particular application (e.g., the size and shape of the device 108 and/or the body part) and may include circular, elliptical, or any other combination of polygonal and arcuate segments.

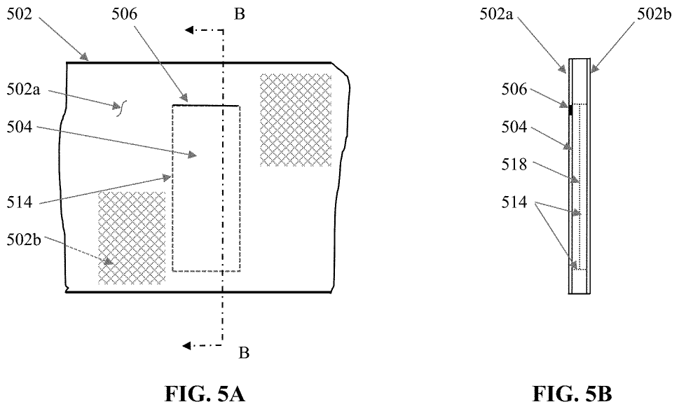

In various embodiments, the envelope 102 is embodied as a single panel, which may comprise one or more layers of material, having an inner surface (closest to the body part) and an outer surface (farthest from the body part), where the receptacle 104 is coupled to either the inner or outer surface to suit a particular application and as described in greater detail with respect to FIGS. 4A and 4B. Alternatively, the envelope 102 can be made of multiple panels that are secured to one another (e.g., by stitching about the edges thereof) and where the receptacle 104 can be disposed between layers, as described in greater detail with respect to FIGS. 5A and 5B.

As further shown in FIG. 1, the device 108 includes a wire or cable 108a extending therefrom that may be used to interface the device 108 with, for example, a computer for data download or a power source for recharging. The garment 100 may include structure to allow the cable 108a to pass therethrough and/or to secure the cable 108a in a particular orientation. For example, the receptacle 104 may include an opening (e.g., 106 or other) that is sized and shaped to accommodate the cable 108a and/or direct the cable 108a away from the patient 110. Furthermore, the garment 100 may include an adjustment mechanism 120, such as, for example, a belt with friction adjustors, an elastic material, or Velcro® to allow the garment 100 to be custom fit to the patient's 110 body part.

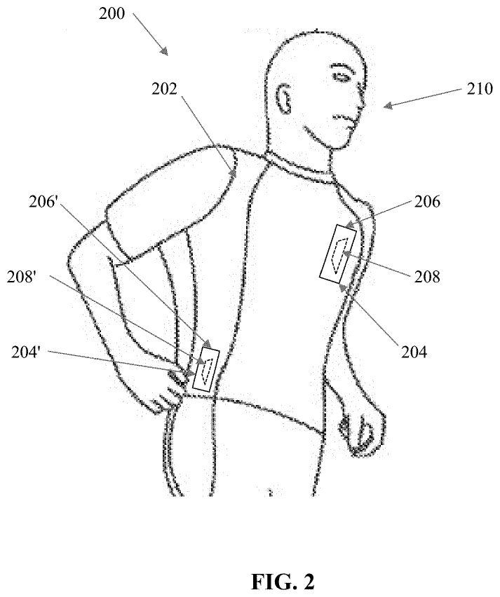

FIG. 2 depicts an alternative garment 200 for interfacing a device 208, 208′ with an implant. The garment 200 is substantially similar to the garment 100 described above insofar as it includes an envelope 202 made of a textile material and includes a receptacle 204 configured with an open end 206 to receive a device 208. The garment 200 shown has an envelope 202 embodied in a vest where the receptacle 204 is oriented to generally align with a patient's 210 torso or chest to, for example, interface with a pacemaker or defibrillator. The garment 200 depicted in FIG. 2 is shown with an alternative or optional receptacle 204′ having an open end 206′ for receiving a device 208′ as described herein. The alternative or optional receptacle 204′ is shown in FIG. 2 for illustrative purposes only; however, in some embodiments, the garments described herein may include multiple receptacles to suit a particular application, such as, for example, multiple devices required for multiple procedures, hold auxiliary equipment (e.g., a battery pack), etc. Additionally, the alternative receptacle 204′ is oriented to generally align with the patient's 210 lower back to for example, interface with a drug delivery device. The garment 200 depicted in FIG. 2 may take the form of a pullover or a vest/shirt type of configuration that includes a closure mechanism (e.g., 312 in FIG. 3) to secure the garment 200 to the patient 210, with or without an adjustment mechanism (e.g., 120 in FIG. 1).

FIG. 3 depicts yet another alternative garment 300 that is also similar in structure to the other garments 100, 200 described herein. The envelope 302 has a generally rectangular and planar shape and is made of a relatively soft/flexible panel that can be wrapped about the patient (not shown), for example, about the midsection of the patient to secure the device 308 proximate to the patient's lower back. However, the overall shape and size of the garment 300 may vary to suit a particular application. The receptacle 304 is shown disposed proximate the midpoint of the envelope 302; however, the exact location of the receptacle 304 may vary to suit a particular application and, in some embodiments, may be removably secured to the envelope 302 (e.g., via Velcro®, snaps, etc.) to allow the receptacle 304, and by extension the device 308, to be positioned as necessary to suit a particular application.

As shown in FIG. 3, the two ends 302a, 302b of the envelope 302 can include an attachment or closure mechanism 312 to secure the garment 300 to the patient. In some embodiments, the mechanism 312 can include two halves 312a, 312b, with one half 312a disposed on/at one end 302a and the second half 312b disposed on/at the other end 302b. In one embodiment, the closure or fastening mechanism 312 includes mating hook and loop fasteners 312a, 312b, each of which may cover a significant portion (e.g., about 50% or more) of its respective end 302a, 302b such that the ends 302a, 302b can overlap and be secured to one another at different positions to provide adjustability to accommodate different size patients. Alternative closure or fastening mechanisms, such as buttons, snaps, buckles, draw strings, zippers, or other mechanisms as would be known to those in the apparel and/or medical clothing industry are contemplated and considered within the scope of the invention.

FIG. 4A is an enlarged view of a portion of a garment and FIG. 4B is a cross-sectional view thereof depicting the receptacle structure 404 in greater detail. As shown in FIG. 4A, the receptacle 404 is disposed on a surface of the envelope 402, which may be an interior or exterior surface of the envelope 402 depending on, for example, the type of garment, the type of electronic device or implant, and/or the location of the implant. The receptacle 404 is attached to the envelope 402 about a perimeter 414 of the receptacle 404 by any of the methods disclosed herein, for example, via stitching. The receptacle 404 includes an open end 406 where the receptacle is not directly attached to the envelope 402, but includes a closure mechanism 416, such as those disclosed herein, for example, Velcro®. The closure mechanism 416 allows the electronic device to be removably secured within the receptacle 404.

FIG. 4B is a cross-sectional view taken at line B-B of FIG. 4A. Generally, the envelope 402 as shown in FIG. 4B is a single panel; however, the envelope may include multiple layers, types of materials, etc. as known to a person of skill in textile manufacturing. The receptacle 404 is attached to the envelope via its perimeter 414 to define an inner cavity 418 with the opening 406 defined by a portion of the perimeter 414 and the envelope 402. The closure mechanism 416 is disposed at the opening 406 is may be attached to one or both of the receptacle 404 or envelope 402 (e.g., two halves of a zipper or a button button-hole combination). The receptacle shown in FIG. 4B appears “pouch-like” so as to illustrate the inner cavity 418; however, the panel(s) that may be used to form the receptacle 404 may lie flat against the envelope so that the electronic device fits snugly within the receptacle, while in other embodiments, the panel(s) may be folded, pleated, etc., so as to create a receptacle with a larger and/or expandable inner cavity 418 as may be required by some applications. Typically, the inner cavity 418 is configured to prevent inadvertent movement of the electronic device, but also allow a certain amount of movement (e.g., via a frictional fit) to allow for adjusting the exact location or orientation of the device within the receptacle to optimize operative alignment between the device and the implant. In various embodiments, the garment may be produced so as to be sterile or aseptic.

FIGS. 5A and 5B depict an alternative garment structure to that shown in FIGS. 4A and 4B, with FIG. 5A being an enlarged view of a portion of a garment and FIG. 5B being a cross-sectional view taken at line B-B of FIG. 5A. As shown in FIG. 5A, the receptacle 504 is disposed within the envelope 502 and is defined by a border 514 (e.g., perimeter) formed within the envelope 502. For example, and as shown in FIG. 5B, the envelope 502 includes a first or front panel 502a and a second or back panel 502b, where the two panels 502a, 502b define a space (e.g., inner cavity 518) therebetween that is further defined by attaching the panels 502a, 502b to one another (e.g., via stitching, bonding, etc.) forming the border or perimeter 514 of the receptacle 504. Generally, the panels 502a, 502b may include multiple layers, types of materials, etc., as known to a person of skill in textile manufacturing, and may be, for example, two separate panels stitched along their edges or a single piece of material folded and/or attached along its longitudinal sides to form a sleeve-like structure, as necessary to suit a particular application. In some embodiments, the back panel 502b comprises an elastic mesh material.

In some embodiments, the receptacle 504 is fully enclosed within the envelope 502 with an opening 506 defined in the first or second panel (e.g., a gap or slit formed in the textile material, with or without a flap or other closure mechanism). In FIG. 5B, the opening 506 is shown in the first panel 502a; however, it could be formed in either or both panels to suit a particular application, such as, for example, accessing the receptacle 504 (and device) via an inner cavity of the garment or an exterior surface of the garment. In addition, the receptacle 504 may include a closure mechanism as described herein. In some embodiments, the opening 406, 506 and/or closure mechanism 416 may include structure for managing cables or other wires running to or from the device. In various embodiments, more than one receptacle 404, 504 may be disposed on and/or within the garment. Additionally, the garment may include indicia (or indicium) applied thereto to provide information to the user/patient, such as letters, numbers, colors, symbols or combinations thereof that function as a warning, a designation of use, indicate a function of a particular component, etc.

Generally, the overall size, shape, and materials of construction of the garment will vary to suit a particular application, for example, the size and type of medical device, intended use, implant location, and/or other functional or aesthetic considerations. In one common variation, the garment is a hat or other type of head piece for interfacing with a brain stimulating implant within the patient's head and may be constructed of stretchable, breathable materials to ensure comfort, flexibility in fit, and durability in active use and in storage.

The various textiles may be made from many different materials, such as animal-based (e.g., wool, silk), plant-based (e.g., cotton, flax, jute, bamboo), mineral-based (e.g., metal or glass fibers), and synthetic (e.g., nylon, polyesters, acrylic, rayon). Electrically conductive woven or knitted fabrics can be incorporated where electromagnetic interference (EMI) shielding properties are desired and include, for example, electrically conductive yarns (carbon fibers, metal fibers), metallization of fabrics or yarns (voltaic, vacuum vaporization); lamination or coating of conductive layers onto the fabric surface with metal particles (e.g., copper-nickel coated polyester), transparent organic metal oxides, carbon, or inherently conductive polymers (ICPs), such as polyaniline, polypyrrole and poly(3,4-ethylenedioxythiophene).

Many modifications and other implementations of the disclosure will come to mind to one skilled in the art to which this disclosure pertains having the benefit of the teachings presented in the foregoing descriptions and the associated figures. Therefore, it is to be understood that the disclosure is not to be limited to the specific implementations disclosed herein and that modifications and other implementations are intended to be included within the scope of the appended claims. Although specific terms are employed herein, they are used in a generic and descriptive sense only and not for purposes of limitation.

What is claimed is:

1. A garment for interfacing an electronic device with a medical implant comprising:
an envelope comprising a textile material and configured to at least partially surround or contact a portion of a human body where the implant is located; and
a receptacle disposed on or within the envelope, wherein the receptacle is configured to removably secure the electronic device to the envelope and in proximity to the portion of a human body where the implant is located.

2. The garment of claim 1, wherein the receptacle is defined by a partially open perimeter.

3. The garment of claim 1, wherein the receptacle is accessible via an interior cavity of the envelope.

4. The garment of claim 1, wherein the receptacle is accessible via an exterior surface of the envelope.

5. The garment of claim 1, wherein the envelope comprises a first panel and a second panel and the receptacle is defined therebetween.

6. The garment of claim 5, wherein the first panel is configured to be in closer proximity to the human body than the second panel, and the second panel defines an opening therethrough configured to receive the electronic device.

7. The garment of claim 6, wherein the first panel comprises an elastic netting.

8. The garment of claim 1, wherein the envelope comprises at least one adjustment mechanism configured to accommodate the garment to differently sized and/or shaped body parts.

9. The garment of claim 1, wherein a portion of the envelope comprises a material configured to allow for the passage of electromagnetic radiation therethrough.

10. The garment of claim 1, wherein a portion of the envelope comprises a material configured to prevent the passage of electromagnetic radiation therethrough.

11. The garment of claim 1, wherein the receptacle is secured to the envelope via at least one of stitching, bonding, hook and loop fastener (Velcro®), buttons, snaps, or zippers.

12. The garment of claim 1, wherein the receptacle is sized and shaped to receive at least one of a receiver, a transmitter, an amplifier, a power source, a digital storage device, a display, or a monitor.

13. The garment of claim 1, wherein the receptacle is configured to provide positional adjustability to the electronic device to provide for a substantially optimal operative alignment with the implant.

14. The garment of claim 1, wherein the envelope is embodied in at least one of a hat, a shirt, a vest, a sleeve, a pair of pants, or a wrap.

15. The garment of claim 1, wherein the envelope comprises at least one attachment mechanism configured to removably secure the garment to a patient.

16. The garment of claim 1, wherein the garment further comprises the electronic device.

17. The garment of claim 16, wherein the electronic device is selected from the group consisting of a receiver, a transmitter, an amplifier, a power source, a digital storage device, a display, or a monitor.

18. A garment for interfacing an electronic device with a medical device implanted within a patient's head, the garment comprising:
an envelope comprising a textile material and configured to at least partially surround the patient's head; and
a receptacle coupled to the envelope and configured to removably secure the electronic device thereto, wherein the receptacle is disposed on or within the envelope so as to operatively align the electronic device with the medical device.

19. The garment of claim 18, wherein the electronic device is a receiver/transmitter for downloading data from the implant and transmitting the data to an intended recipient.

20. A method of interfacing an electronic device with a medical implant disposed within a body, the method comprising the steps of:
providing a garment comprising a textile material and configured to at least partially surround a portion of the body, wherein the garment includes a receptacle configured to removably secure the electronic device at least partially therein;
disposing the garment about the body; and
orienting the garment relative to the body portion where the implant is located to operatively align the electronic device with the medical device.
